(19) United States
(12) Patent Application Publication
(10) Pub. No.: US 2010/0217272 A1
(43) Pub. Date: Aug. 26, 2010

(54) Method and apparatus for positioning implant between spinous processes

(75) Inventors: David B. Baughman, Louisville, KY (US); Richard B. Holt, Prospect, KY (US)

Correspondence Address: NIXON & VANDERHYE, PC, 901 NORTH GLEBE ROAD, 11TH FLOOR, ARLINGTON, VA, 22203, US

(73) Assignee: Holt Development LLC, Louisville, KY (US)

(21) Appl. No.: 12/379,441

(22) Filed: Feb. 20, 2009

(51) Int. Cl. A61B 17/58

(52) U.S. Cl. 606/99

(57) ABSTRACT

A method and apparatus for positioning an implant between spinous processes, generally comprising a cannula for percutaneous insertion between the spinous processes, an obturator that can be removably mounted within the cannula and is slidable therein so that an inner end thereof can moved inwardly beyond an inner end of the cannula, and an implant that is constructed to be movable within the cannula when the obturator is removed therefrom for positioning between the spinous processes. In accordance with the method, an insertion is made through the skin on one side of the rear portion of the spine, the cannula having the obturator slidably movable therein is inserted inwardly through the incision so that the inner end of the cannula is positioned between the spinous processes, the obturator is removed from the cannula, the implant is inserted within the cannula and moved therein to a position between the spinous processes, and the cannula is removed from the implant. The implant may be retained between the spinous processes when the cannula is removed therefrom by inflating the implant or mechanically expanding it.

### BACKGROUND OF THE INVENTION

1. Field of the Invention

The present invention relates to a method and apparatus for positioning an implant between spinous processes and, more particularly, to such a method and apparatus which can be utilized in a minimally invasive percutaneous surgical procedure.

2. Description of Related Art

Spinal stenosis is a narrowing of the spinal canal which can lead to pain, weakness and/or numbness when the narrowing leads to compression of the spinal cord and nerve roots. The nerves react by swelling and undergoing inflammation.

Although some people are born with spinal stenosis, more typically it occurs as the gradual result of aging and deterioration of the spine as a result of everyday activities. The incidents of spinal stenosis increase as people grow older.

It is possible to treat stenosis without surgery, e.g., through the use of medications, injections, rest or restricted activity, or physical therapy. In cases where non-surgical treatments have not been effective, surgical treatments have been performed which, though effective in certain instances, are invasive and thus subject the patient to certain risks. The use of surgically implanted devices that distend adjacent spinous processes have also involved invasive procedures with consequent risks to the patient.

The present invention is not subject to the disadvantages of the previously used surgical and non-surgical procedures, and possesses significant advantages over the previously used procedures.

### BRIEF SUMMARY OF THE INVENTION

The new and improved method and apparatus for positioning an implant between spinous processes in accordance with the present invention can be utilized in minimally invasive percutaneous surgical procedures, thereby minimizing the risks of the patient.

In accordance with the present invention, an incision is made on one side of the spine in a location to have access to the space created between the spinous processes in which the implant is to be positioned. In one embodiment, an elongated, curved cannula is inserted through the incision on one side of the spine and positioned between the spinous processes. An elongated obturator is slidably removed within the cannula and has a sharp inner end extending beyond the inner end of the cannula to facilitate its insertion and positioning. The outer end of the obturator has a handle portion to facilitate is removal from the cannula. When the cannula is in the desired position between the spinous processes, the obturator is slidably removed therefrom to allow access to the inner portion of the cannula from the outer end thereof.

An implant is positioned in the cannula and pushed into a position extending out of the inner end thereof between the spinous processes. The obturator may be used to push the implant into the desired position. The implant is provided with an inner portion that expands or can be inflated or enlarged to retain the implant in a desired position between the spinous processes when the cannula is then removed from between the spinous processes. After removal of the cannula, the implant, in one embodiment, is then inflated or enlarged to its desired shape between the spinous processes by filling it with bone cement, polyurethane or another suitable material. In another embodiment, the implant may have folded or collapsed end portions that can be mechanically expanded to retain it in the desired position between the spinous processes when the cannula is removed therefrom.

### BRIEF DESCRIPTION OF THE DRAWINGS

FIG. 1 is a schematic view in perspective of adjacent spinous processes having a space therebetween in which an implant is to be positioned;

FIG. 2 is an elevational view of a portion of a person's back showing an incision on one side of the spine in accordance with one method of the present invention;

FIG. 3 is a perspective view in partially schematic form of a curved cannula/obturator inserted through the incision made on one side of the spine, as shown in FIG. 2, and positioned between the spinous processes;

FIG. 4 is a perspective view like FIG. 3 showing the inner obturator removed from the cannula;

FIG. 5 is a perspective view like FIG. 4 showing an implant slidably inserted through the cannula positioned between the spinous processes and extending out of the inner end of the cannula;

FIG. 6 is a view similar to FIG. 5 showing the inner end of the implant being inflated or enlarged to retain it in position between the spinous processes when the cannula is removed therefrom;

FIG. 7 is a perspective view like FIG. 6 showing the inner end of the implant being inflated to retain it in position between the spinous processes when the cannula is removed therefrom;

FIG. 8A is a perspective view of one embodiment of an implant that is stretched to a narrow shape so that it can be inserted through a cannula between the spinous processes;

FIG. 8B is a perspective view of the implant shown in FIG. 8A after the tension is removed therefrom so that it assumes its normal shape;

FIG. 9 is a perspective view like FIG. 7 showing the implant positioned between the spinous processes and fully inflated after the cannula is removed therefrom;

FIG. 10 is a perspective view of another embodiment of an implant positioned between the spinous processes before it is mechanically actuated to retain it in position;

FIG. 11 is a perspective view like FIG. 10 showing the implant after it is mechanically actuated to retain it in position between the spinous processes.

### DETAILED DESCRIPTION OF THE INVENTION

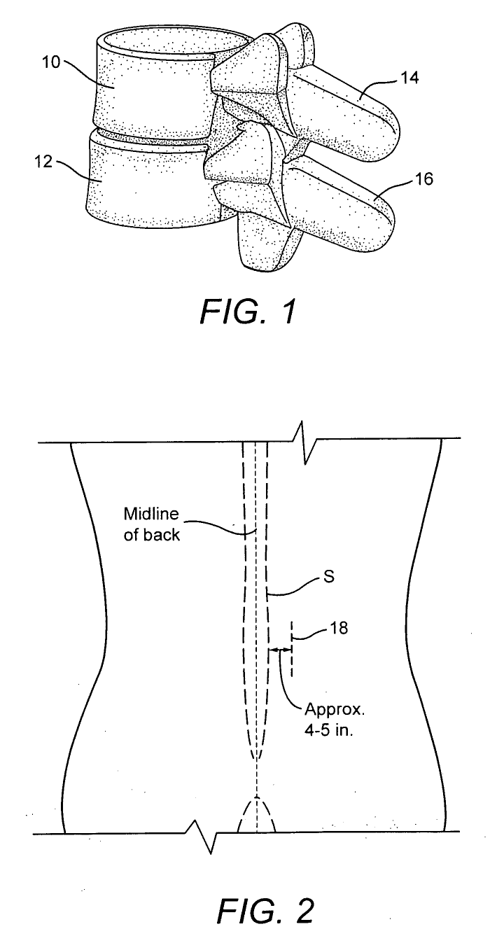

FIG. 1 illustrates schematically a pair of adjacent vertebral bodies 10, 12 of the spine each having a rearwardly facing spinous process 14, 16, respectively. The present invention is directed to a method and apparatus for inserting an implant between the spinous processes 14, 16 for the purpose of increasing the spacing therebetween to relieve pain or other symptoms caused by spinal stenosis or the like.

In accordance with one embodiment of the present invention, an incision 18 is made approximately 4-5 inches on one side of the spine S, as shown in FIG. 2. The incision 18 may be of any suitable length, such as 0.75 inches. In this manner, a cannula and an implant of a suitable size can be positioned between the spinous processes 14, 16 by non-invasive percutaneous access through the incision 18 which is located in close proximity to the spine.

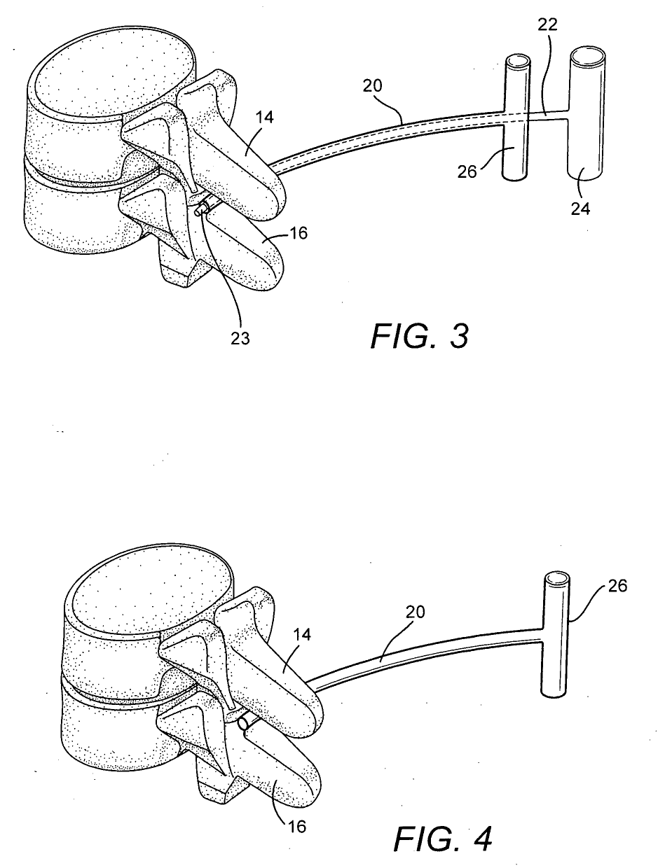

As shown schematically in FIG. 3, a cannula 20 with an inner obturator 22 slidably mounted therein and having a sharp inner end 23 extending out of the inner end of the cannula 20 is inserted into the incision 18 and positioned between the spinous processes 14, 16 with the use of any suitable imaging guidance or the like. After positioning of the cannula 20, the inner obturator 22 is removed therefrom as shown in FIG. 4 by grasping its outer handle 24 to allow access to the space between spinous processes through the cannula. As an illustrative embodiment, the cannula 20 may be approximately ¼ of an inch in diameter and may have a radius of curvature of approximately 6 inches. The cannula 20 is provided with an outer handle 26.

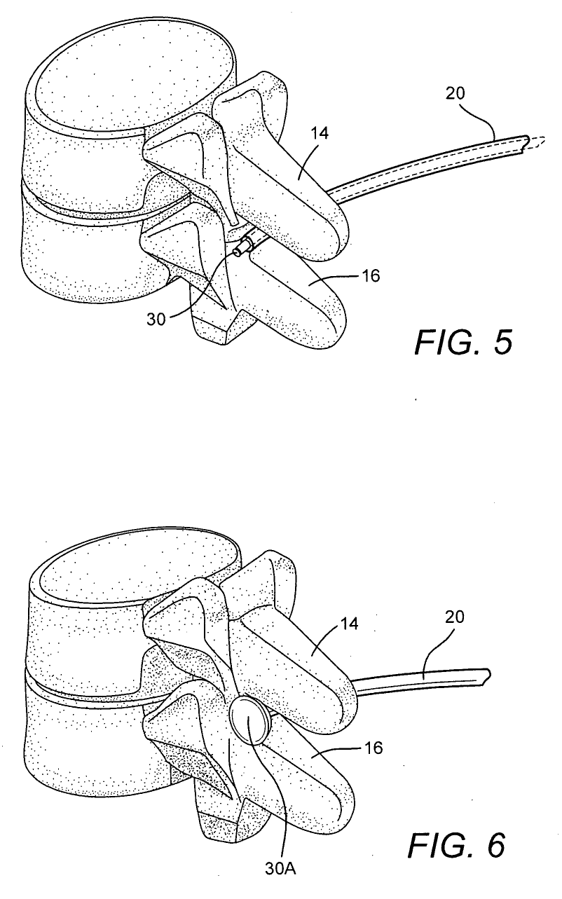

As shown in FIG. 5, an implant 30 is then loaded into the cannula 20 and positioned between the spinous processes 14, 16. The implant 30 is pushed through the cannula into a position wherein it extends out of the inner end of the cannula 20, as shown in FIG. 5.

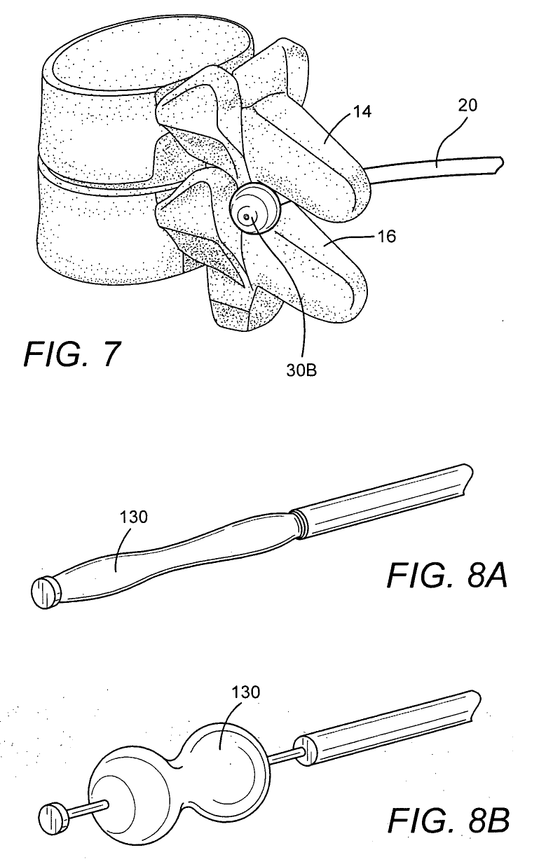

In order to hold the implant 30 in place when the cannula 20 is removed therefrom between the spinous processes, the implant 30 may have a mechanically expandable inner end 30A in the form of an umbrella-type device shown in FIG. 6 or the inner end of the implant 30 may have an inflatable portion 30B as shown in FIG. 7. In the embodiment of FIG. 7, the inner end 30B of the implant is approximately one-half of the length of the implant and is inflated by filling it with a suitable substance such as a bone cement, polyurethane or the like. Thereafter, the cannula 20 can be removed from the implant and the implant can be fully inflated by filling it with bone cement or the like to retain it in the desired position between the spinous processes, as shown in FIGS. 9 and 9A with respect to the implant 30B. An advantage of filling a flexible implant with a bone cement or other cement-like material is that a custom fit can be accomplished between the spinous processes so that the forces become spread over more bone area to avoid any detrimental impact from point loading. It is noted that the implant may have any suitable shape when inflated which will serve to retain it between the spinous processes.

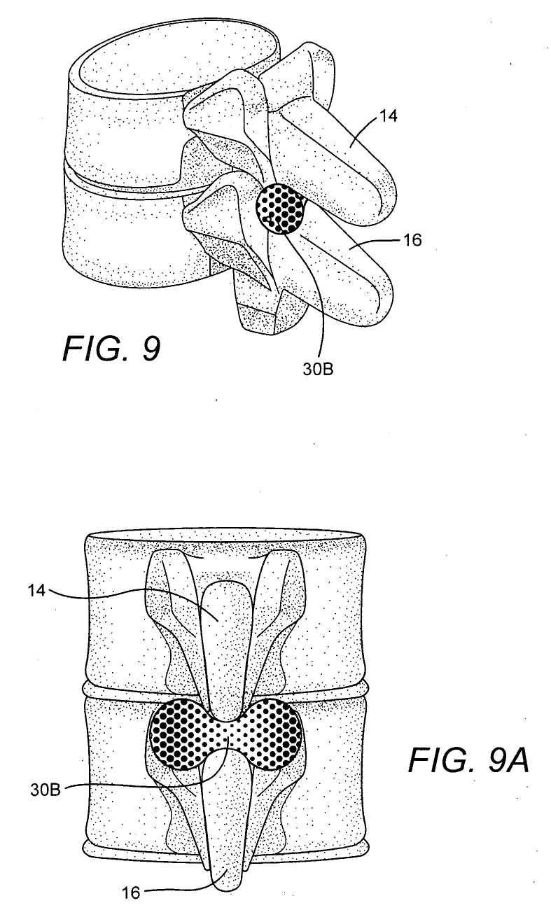

FIGS. 8A and 8B disclose another embodiment of an implant 130 that is flexible and hollow such that it can be stretched as shown in FIG. 8A to insert it through the cannula 20 into the desired position between the spinous processes. Thereafter, the tension is reduced to allow the implant 130 to return to its natural shape as shown in FIG. 8B between the spinous processes. The implant 130 can then be filled with a suitable bone cement or the like.

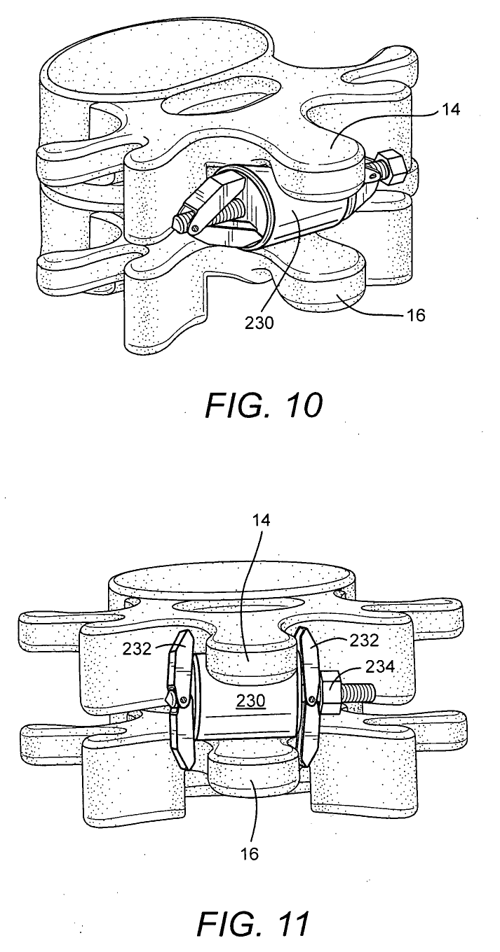

FIGS. 10 and 11 illustrate a further embodiment of an implant 230 that can be mechanically actuated to retain it in position after it has been inserted through a cannula into the desired position between the spinous processes 14, 16. After the implant 230 is in the desired position, it is mechanically actuated in any suitable manner to cause arms 232 at both ends thereof to open or expand outwardly to retain the implant 230 in position between the spinous processes. A nut 234 or other locking means may be used to lock the arms 232 in their deployed state, as shown in FIG. 11.

As illustrative embodiments, the cannula 20 and obturator 22 may be formed of any suitable material, such as aluminum, titanium, stainless steel or a carbon fiber composite; and the implant 30, 30A, 30B, 130 and 230 may be formed of any suitable material, such as polyurethane, PEEK, Teflon, UHMW, polyethylene, titanium or stainless steel. The implants may be inflated by any suitable type of bone cement, such as methyl methacrylate, or by any other suitable material such as polyurethane, silicon or saline.

From the foregoing description, it will be readily seen that the cannula and obturator of the method and apparatus of the present invention facilitate the positioning of an implant between spinous processes in a simple and efficient manner through non-invasive percutaneous access positioned close to the spine to avoid interference with other body portions near the spine. Also, the construction of the implants facilitates the proper installation and positioning thereof.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. Apparatus for positioning an implant between spinous processes comprising:
a cannula for percutaneous insertion between the spinous processes;
an obturator that is removably mounted within said cannula and slidable therein so that an inner end thereof can be moved inwardly beyond an inner end of said cannula; and
an implant that is constructed to be movable within said cannula when said obturator is removed therefrom for positioning between the spinous processes.

2. The apparatus of claim 1 wherein said implant is constructed to be slidably movable within said cannula.

3. The apparatus of claim 2 wherein said implant is inflatable to retain it in position between the spinous processes when separated from said cannula.

4. The apparatus of claim 2 wherein said implant is mechanically expandable to retain it in position between the spinous processes when separated from said cannula.

5. The apparatus of claim 3 wherein said implant is formed of a flexible and resilient material.

6. The apparatus of claim 5 wherein said implant can be elongated under tension to fit within said cannula and between the spinous processes, and said implant is constructed to return to a normal expanded shape between the spinous processes when the tension is removed therefrom and it is separated from said cannula.

7. The apparatus of claim 1 wherein said obturator has a sharp inner end portion and has a handle on an outer end thereof.

8. The apparatus of claim 1 wherein said cannula is curved in shape and has a handle on an outer end thereof.

9. The apparatus of claim 8 wherein said obturator is curved in shape so that it can be slidably inserted within said cannula.

10. The apparatus of claim 8 said obturator is flexible so that it can be slidably inserted within said cannula.

11. A method of positioning an implant between spinous processes, comprising:
making an incision through the skin on one side of the rear portion of the spine;
inserting a cannula having an obturator slidably movable therein and extendable out of an inner end thereof inwardly through the incision so that the inner end of said cannula is positioned between the spinous processes;
removing said obturator from said cannula;
inserting an implant within said cannula and moving it therein to a position between the spinous processes; and
removing said cannula from said implant.

12. The method of claim 11 wherein said implant is slidably movable within said cannula so that it can be pushed to a position between the spinous processes.

13. The method of claim 11 wherein said implant is inflatable, and further comprising inflating said implant to retain it in position between the spinous processes when the cannula is removed therefrom.

14. The method of claim 13 wherein said implant is inflated with a bone cement or polyurethane.

15. The method of claim 11 further comprising moving an inner end of said implant within said cannula so that it extends inwardly beyond an inner end of said cannula;
and expanding the inner end of said implant to retain it in position between the spinous processes when said cannula is removed therefrom.

16. The method of claim 15 wherein said implant is inflatable and the inner end thereof is expanded by inflating it.

17. The method of claim 15 wherein the inner end of said implant is mechanically expanded.

18. The method of claim 11 wherein said implant is elongated under tension from a normal expanded shape so that it can be fit within said cannula, and said implant returns to the normal expanded shape to retain it between the spinous processes when the cannula is removed therefrom.

19. The method of claim 11 wherein said implant is mechanically expandable, and further comprising expanding said implant to retain it in position between the spinous processes when the cannula is removed therefrom.

20. The method of claim 19 wherein the end portions of said implant are expandable.
